(19) United States
(12) Patent Application Publication
(10) Pub. No.: US 2026/0020801 A1
(43) Pub. Date: Jan. 22, 2026

(54) METHOD FOR PREDICTING DELIRIUM OCCURRENCE, AND DEVICE FOR PREDICTING DELIRIUM OCCURRENCE USING SAME

(71) Applicants: INDUSTRY-ACADEMIC COOPERATION FOUNDATION, YONSEI UNIVERSITY, Seoul (KR); HUMAN ANTI-AGING STANDARDS RESEARCH INSTITUTE CO., LTD, Gyeongsangnam-do (KR)

(72) Inventors: Bon Nyeo KOO, Seoul (KR); Jung Mi CHOI, Gyeongsangnam-do (KR)

(73) Assignees: INDUSTRY-ACADEMIC COOPERATION FOUNDATION, YONSEI UNIVERSITY, Seoul (KR); HUMAN ANTI-AGING STANDARDS RESEARCH INSTITUTE CO., LTD, Gyeongsangnam-do (KR)

(21) Appl. No.: 18/875,398

(22) PCT Filed: May 15, 2023
(86) PCT No.: PCT/KR2023/006528

§ 371 Date: Dec. 16, 2024

(30) Foreign Application Priority Data
Jun. 17, 2022 (KR) 10-2022-0074205

(51) Int. Cl. A61B 5/16; A61B 5/00; A61B 5/369

(52) U.S. Cl. CPC A61B 5/165; A61B 5/369; A61B 5/7275

(57) ABSTRACT

The present specification provided a method and device for providing information on delirium occurrence, wherein the information is provided prior to surgery. The method, which is implemented by a processor, comprises the steps of: receiving an electroencephalogram (EEG) signal obtained from an individual prior to surgery; and predicting the likelihood of postoperative delirium occurrence based on the received EEG signal.

### CROSS REFERENCE TO RELATED APPLICATION(S)

This application is a 35 U.S.C. § 371 National Phase Entry Application from PCT/KR2023/006528, filed on May 15, 2023, which claims the benefit of Korean Patent Application No. 10-2022-0074205, filed on Jun. 17, 2022, the disclosures of which are herein incorporated by reference in their entirety.

### TECHNICAL FIELD

The present disclosure relates to a method for predicting delirium occurrence and a device for predicting delirium occurrence using the same.

### BACKGROUND ART

Delirium is one of the most common psychiatric disorders in the elderly after surgery. Such a postoperative delirium (POD) frequently occurs in the elderly and is associated with a high mortality rate.

Delirium is usually diagnosed through an interview about changes in consciousness or orientation by means of observation of symptoms and medical examination. In the case of the confusion assessment method (CAM) which is the most common diagnostic test for delirium, there is no standardized diagnostic method, the cause is multifactorial, and there is no treatment. Therefore, consistent and close observation and interviews by medical staff are necessary for an acute onset, a fluctuating course of a mental status, inattention, disorganized thinking, and an altered level of consciousness.

The electroencephalogram (EEG, brainwave) is an electrophysiological monitoring method which records electrical activity of the scalp and is used to evaluate a brain function by representing macroscopic activity of a brain surface. The EEG is noninvasive, by normally placing electrodes along a scalp and is not affected by the patient's cultural background and education level, and an examiner's skill level, unlike the interview-based neuropsychological tests. Further, the EEG is attracting attention for its usefulness as a marker for cognitive impairment and dementia.

The background of the present disclosure is described for easier understanding of the present disclosure. It should not be understood to admit the matters described in the background of the present disclosure as a prior art.

### DETAILED DESCRIPTION OF THE INVENTION

### Technical Problem

When delirium is diagnosed, it is important to distinguish risk groups in advance and make an early diagnosis. That is, for early diagnosis, the likelihood of occurrence is predicted to be observed closely. In particular, when the patient was old, had many comorbid diseases, had a preexisting central nervous system disease, or especially had a pretexting cognitive impairment, the likelihood of developing delirium was found to be high. Therefore, it is important to determine in advance whether the patient has cognitive impairment in the early diagnosis of delirium.

The cognitive impairment is determined based on several questionnaires. Normally, the questionnaire used for dementia diagnosis is mainly used. However, there is a disadvantage in that if the patient has a short period of education and thus has poor comprehension skills, the patient may be diagnosed with cognitive impairment.

In the meantime, the inventors recognized that when the cognitive function was measured by the EEG based test, these subjective problems may be ruled out and a preoperative EEG measurement value of patients who developed postoperative delirium (delirium group) was different from a preoperative EEG measurement value of patients who did not develop postoperative delirium (non-delirium group).

Accordingly, the inventors recognized that the occurrence of delirium could be predicted with only two EEG channels and more particularly, the likelihood of postoperative delirium occurrence could be predicted using a MDF value derived from EEG prior to a surgery.

In the existing EEG, 64 channels are mainly used. The existing EEG equipment uses electrodes attached to measure a brain signal, which causes inconvenience and cumbersome of a user.

Accordingly, an object to be achieved by the present disclosure is to provide a delirium occurrence predicting method and a delirium occurrence predicting device to predict a likelihood of postoperative delirium occurrence based on an EGG signal received from an individual prior to a surgery.

Objects of the present disclosure are not limited to the above-mentioned objects, and other objects, which are not mentioned above, may be clearly understood by those skilled in the art from the following descriptions.

### Technical Solution

In order to achieve the objects as described above, a delirium occurrence predicting method according to an exemplary embodiment of the present disclosure is provided. The delirium occurrence predicting method according to an exemplary embodiment of the present disclosure is a predicting method for delirium occurrence implemented by a processor and includes the steps of receiving an electroencephalogram (EEG) signal obtained from an individual prior to a surgery; and predicting a likelihood of postoperative delirium occurrence based on a received EEG signal.

According to still another feature of the present disclosure, the step of predicting a likelihood of postoperative delirium occurrence based on a received EEG signal includes a step of predicting a likelihood of postoperative delirium occurrence based on a power value between 5.5 to 13 Hz, of the EEG signal, at the resting with eyes closed.

According to still another feature of the present disclosure, the step of predicting a likelihood of postoperative delirium occurrence based on a received EEG signal may further include a step of predicting a likelihood of postoperative delirium occurrence based on a median dominant frequency (MDF) of power values in a frequency band between 5.5 to 13 Hz, of the EEG signal, at the resting with eyes closed.

According to still another feature of the present disclosure, the step of predicting a likelihood of postoperative delirium occurrence based on a received EEG signal may further include a step of predicting that the likelihood of postoperative delirium occurrence is high when the MDF value is 8.40 or lower.

According to still another feature of the present disclosure, in the step of receiving an EEG signal obtained from an individual prior to a surgery, the EEG signal may be obtained through only two EEG channels.

According to still another feature of the present disclosure, two EEG channels may be located on a prefrontal cortex of the individual.

According to still another feature of the present disclosure, the surgery may be a surgery which requires general anesthesia.

In order to achieve the objects as described above, a delirium occurrence predicting device according to an exemplary embodiment of the present disclosure is provided.

At this time, the delirium occurrence predicting device includes a communication unit configured to receive an EEG signal obtained from an individual prior to a surgery; and a processor connected to the communication unit, and the processor is configured to predict a likelihood of postoperative delirium occurrence based on a received EEG signal.

According to a feature of the present disclosure, the processor may be configured to predict the likelihood of postoperative delirium occurrence based on a power value of a frequency range of 5.5 to 13 Hz in the EEG signal.

According to another feature of the present disclosure, the processor may further be configured to predict the likelihood of postoperative delirium occurrence based on a median dominant frequency (MDF) of power values in a frequency band of 5.5 to 13 Hz in the EEG signal.

According to still another feature of the present disclosure, the processor may be configured to predict that when the MDF value is 8.40 or lower, the likelihood of postoperative delirium occurrence is high.

According to still another feature of the present disclosure, the communication unit may further be configured to obtain an EEG signal obtained from the individual prior to the surgery through only two EEG channels.

According to still another feature of the present disclosure, the two EEG channels may be configured to be located on a prefrontal cortex of the individual.

According to still another feature of the present disclosure, the surgery may be a surgery which requires general anesthesia.

### Effects of the Invention

According to the present disclosure, the likelihood of postoperative delirium occurrence can be predicted with an EEG signal obtained from a prefrontal cortex site through only two EEG channels prior to a surgery and thus early delirium prevention strategies can be provided to the patient and information about whether to proceed with the surgery can be provided. To be more specific, according to the present disclosure, two-channel EEG is used, rather than 64-channel EEG which is widely used in the related art, so that a cost for developing a device for diagnosing delirium prior to the surgery is saved.

In addition, according to the present disclosure, the likelihood of the postoperative delirium occurrence can be predicted in advance only with a median dominant frequency (MDF) value of power values in a frequency band between 5.5 Hz to 13 Hz, in the EEG signal of an individual prior to the surgery.

The effects according to the present disclosure are not limited to the contents exemplified above, and more various effects are included in the present specification.

### BRIEF DESCRIPTION OF DRAWINGS

FIG. 1A is a schematic view for explaining a delirium occurrence predicting system using a delirium occurrence predicting method, according to an exemplary embodiment of the present disclosure.

FIG. 1B is a schematic view for explaining a delirium occurrence predicting device according to an exemplary embodiment of the present disclosure.

FIG. 1C is a schematic view for explaining a medical staff device which is a component of a delirium occurrence predicting system according to an exemplary embodiment of the present disclosure.

FIG. 2 illustrates a procedure of a delirium occurrence predicting method according to an exemplary embodiment of the present disclosure.

FIG. 3 illustrates a delirium group and a non-delirium group predicted using a delirium occurrence predicting method and device according to an exemplary embodiment of the present disclosure.

FIG. 4 illustrates information and evaluation results of a delirium group and a non-delirium group predicted using a delirium occurrence predicting method and device according to an exemplary embodiment of the present disclosure.

FIG. 5 illustrates a univariate regression analysis result on a probability of postoperative delirium occurrence of assessment results of a delirium group and a non-delirium group predicted using a delirium occurrence predicting method and device according to an exemplary embodiment of the present disclosure.

FIG. 6 illustrates a multivariate regression analysis result on a probability of postoperative delirium occurrence of assessment results of a delirium group and a non-delirium group predicted using a delirium occurrence predicting method and device according to an exemplary embodiment of the present disclosure on a meta-analysis forest plot.

FIG. 7A illustrates a median dominant frequency (MDF) value result of power values in an EEG frequency band between 5.5 Hz and 13 Hz of one individual predicted using a delirium occurrence predicting method and device according to an exemplary embodiment of the present disclosure.

FIG. 7B illustrates a delirium occurrence predicting information providing screen of an individual predicted using a delirium occurrence predicting method and device according to an exemplary embodiment of the present disclosure.

FIG. 8A illustrates a median dominant frequency (MDF) value result of power values in an EEG frequency band between 5.5 Hz and 13 Hz of another individual predicted using a delirium occurrence predicting method and device according to an exemplary embodiment of the present disclosure.

FIG. 8B illustrates a delirium occurrence predicting information providing screen of another individual predicted using a delirium occurrence predicting method and device according to an exemplary embodiment of the present disclosure.

### BEST MODE FOR CARRYING OUT THE INVENTION

Advantages and characteristics of the present disclosure, and a method of achieving the advantages and characteristics will be clear with reference to an exemplary embodiment described in detail together with the accompanying drawings. However, the present disclosure is not limited to the following exemplary embodiments but may be implemented in various different forms. The exemplary embodiments are provided only to complete disclosure of the present disclosure and to fully provide a person having ordinary skill in the art to which the present disclosure pertains with the category of the disclosure. With regard to the description of drawings, like reference numerals denote like components.

In this specification, the terms “have”, “may have”, “include”, or “may include” represent the presence of the characteristic (for example, a numerical value, a function, a surgery, or a component such as a part”), but do not exclude the presence of additional characteristic.

The terms “configured to (or set to)” may be interchangeably used with “suitable for”, “having the capacity to”, “designed to”, “adapted to”, “made to”, or “capable of” depending on the situation. The terms “configured to (or set)” may not necessarily mean only “specifically designed to” in a hardware manner. Instead, in some situations, the terms “a device configured to” may mean that the device is capable of something together with another device or components”. For example, the terms “a processor configured (or set) to perform A, B, and C” may refer to a dedicated processor (for example, an embedded processor) configured to perform the corresponding surgery or a generic-purpose processor (for example, a CPU or an application processor) which is capable of perform the surgeries by executing one or more software programs stored in a memory device.

The terms used in this specification are merely used to describe a specific embodiment, but do not intend to limit the scope of another embodiment. A singular form may include a plural form if there is no clearly opposite meaning in the context. Terms used herein including technical or scientific terms may have the same meaning as commonly understood by those skilled in the art. Among the terms used in this specification, terms defined in the general dictionary may be interpreted as having the same or similar meaning as the meaning in the context of the related art, but is not ideally or excessively interpreted to have formal meanings unless clearly defined in this specification. In some cases, even though the terms are defined in this specification, the terms are not interpreted to exclude the embodiments of the present specification.

The features of various embodiments of the present disclosure may be partially or entirely bonded to or combined with each other and may be interlocked and operated in technically various ways understood by those skilled in the art, and the embodiments may be carried out independently of or in association with each other.

For clarity of interpretation of the present specification, terms used in the present specification will be defined below.

The term used in the specification, “delirium”, is one of common psychiatric disorders in the elderly and refers to temporary and very sudden confusion of mental status. To be more specific, delirium in the specification may refer to a delirium which may occur after the surgery, that is, “postoperative delirium (POD)”.

The term used in the specification, “individual”, may be an individual who is diagnosed by a medical staff to have a likelihood of a postoperative delirium occurrence using the method and the device of the present disclosure. Desirably, in the specification, the individual may be a subject, or a patient about to undergo a surgery, and more desirably, a patient about to undergo a surgery under general anesthesia, and still more desirably, a patient over 70 years of age with a high likelihood of delirium occurrence about to undergo a surgery under general anesthesia.

The term used in the specification, a signal (electroencephalogram signal)” may refer to an EEG signal value recorded in a sensor which senses a brainwave. To be more specific, the EEG signal may be acquired by measuring an electrical signal generated from the brain, using two or more electrode channels.

In the meantime, the EEG signal may be a signal or a signal value acquired from a sensor so that in the present specification, the EEG signal may be interpreted as the same meaning as brainwave data.

According to the feature of the present disclosure, the EEG signal may be time-series brainwave data acquired in a resting state in which stimulation is not applied to the individual, but is not limited thereto.

The term used in the specification, “median dominant frequency (abbreviated as “MDF)” is a marker which describes a phenomenon that brain rhythms slow down when the individual closes the eyes, in the EEG signals obtained from the individual and in the specification, refers to a median dominant frequency (MDF) of power values in the frequency band between 5.5 Hz and 13 Hz. Further, MDF in the specification may also be called peak median edge frequency (peak-MEF).

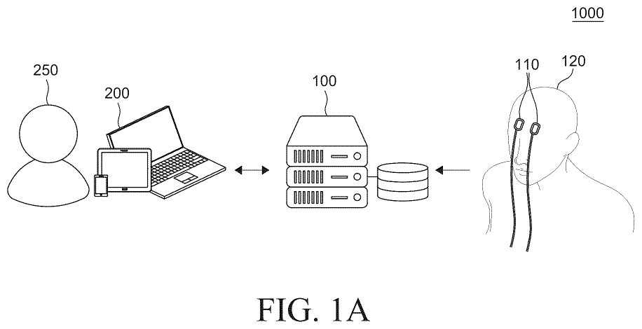

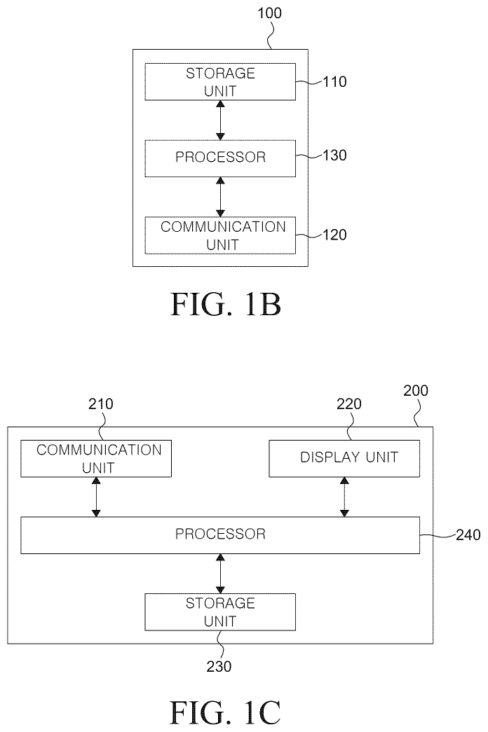

Hereinafter, a delirium occurrence predicting device using a delirium occurrence predicting method according to an exemplary embodiment of the present disclosure will be described in detail with reference to FIGS. 1A to 1C.

FIG. 1A is a schematic view for explaining a delirium occurrence predicting system using a delirium occurrence predicting method, according to an exemplary embodiment of the present disclosure.

Referring to FIG. 1A, a delirium occurrence predicting system 1000 may be a system configured to provide postoperative delirium occurrence prediction data based on a brainwave of an individual prior to a surgery.

Further, the delirium occurrence predicting system 1000 may be configured by a delirium occurrence predicting device 100 configured to predict whether delirium occurs in an individual based on an EEG signal and a medical staff device 200.

The delirium occurrence predicting device 100 includes only two EEG channels 105 and interworks with the medical staff device 200. Further, the delirium occurrence predicting device 100 receives EEG signals from two EEG channels 105 and extracts a feature from the received EEG signals to provide data for predicting delirium occurrence to the medical staff device 200. In order to configure the EEG channel 105, two electrodes and an additional ground electrode may be demanded.

The delirium occurrence predicting device 100 provides the delirium occurrence prediction data as a web page through a web browser installed in the medical staff device 200 or may provide the data as an application or a program. In various exemplary embodiments, the information may be provided to be included in a platform in a client-server environment.

Next, components of the delirium occurrence predicting device 100 of the present disclosure will be described in detail with reference to FIG. 1B.

FIG. 1B is a schematic view for explaining a delirium occurrence predicting device according to an exemplary embodiment of the present disclosure.

Referring to FIG. 1B, the delirium occurrence predicting device 100 includes a storage unit 110, a communication unit 120, and a processor 130. The communication unit 120 configured to receive an EEG signal obtained from an individual prior to a surgery is connected to the processor 130 and the processor may be configured to predict a likelihood of postoperative delirium occurrence based on the EEG signal received from the EEG channel 105.

The storage unit 110 may store various data to evaluate the likelihood of postoperative delirium occurrence for an individual, prior to a surgery. In the exemplary embodiment, the storage unit 110 may include at least one storage medium of a flash memory type, a hard disk type, a multimedia card micro type, a card type memory (for example, an SD or XD memory), a RAM, an SRAM, a ROM, an EEPROM, a PROM, a magnetic memory, a magnetic disk, and an optical disk.

The communication unit 120 connects the delirium occurrence predicting device 100 to be communicable with the external device. The communication unit 120 is connected to the medical staff device 200 using wired/wireless communication to transmit and receive various data. Specifically, the communication unit 120 receives EEG signals of an individual from the EEG channel 105 and may receive the EEG signal from a brain electromagnetic tomography (not illustrated). Further, the communication unit 120 may transmit an analysis result to the medical staff device 200.

The processor 130 is operatively connected to the storage unit 110 and the communication unit 120 and may perform various instructions to analyze the EEG signal for the individual.

Specifically, the processor 130 receives an EEG signal of the individual from the EEG channel 105 through the communication unit 120 and extracts the MDF value based on the received EEG signal to predict the likelihood of postoperative delirium occurrence for the individual.

Moreover, the processor 130 may provide delirium occurrence prediction data based on feature data including the MDF value extracted from the EEG signal.

In various exemplary embodiments, the processor 130 may use a prediction model configured to predict a likelihood of postoperative delirium occurrence based on at least one feature data, among an MDF extracted from the EEG signal, power spectrum densities (PSDs), a functional connectivity, and a network index. The processor 130 inputs the feature data from the EEG signal to the prediction model as an input to output a likelihood of postoperative delirium occurrence.

In various exemplary embodiments, the processor 130 may predict the likelihood of postoperative delirium occurrence based on the median dominant frequency (MDF) of power values in the frequency range of the EEG signal of 5.5 to 13 Hz in a step of predicting the likelihood of postoperative delirium occurrence based on the received EEG signal.

To be more specific, the processor 130 may predict the likelihood of postoperative delirium occurrence based on the median dominant frequency (MDF) of power values in the frequency band of the EEG signal between 5.5 Hz and 13 Hz in a step of predicting the likelihood of postoperative delirium occurrence based on the received EEG signal.

To be more specific, when the MDF value is 8.40 or lower in the step of predicting the likelihood of postoperative delirium occurrence based on the received EEG signal, the processor 130 may predict that the likelihood of postoperative delirium occurrence is high.

In the step of receiving an EEG signal obtained from an individual prior to a surgery, the communication unit 120 may obtain the EEG signal through only two channels.

Together with this, two channels which obtain the EEG signal may be located on the prefrontal cortex of the individual.

Next, referring to FIG. 1C, a component of the medical staff device 200 which is a component of the delirium occurrence predicting device of the present disclosure will be described.

FIG. 1C is a schematic view for explaining a medical staff device which is a component of a delirium occurrence predicting system according to an exemplary embodiment of the present disclosure.

Referring to FIG. 1C, the medical staff device 200 includes a communication unit 210, a display unit 220, a storage unit 230, and a processor 240.

Referring to FIG. 1C, a medical staff may easily obtain information about the likelihood of postoperative delirium occurrence of an individual by means of the medical staff device 200 and also after the surgery, compares and analyzes the preoperative EEG signal through the EEG signal and the analysis result obtained by the delirium occurrence predicting method of the present disclosure and the delirium occurrence predicting device using the method to diagnose the postoperative delirium onset.

As described above, according to the present disclosure, the likelihood of postoperative delirium occurrence is provided prior to the surgery to contribute to early diagnosis of postoperative delirium occurrence and good treatment prognosis.

The communication unit 210 connects the medical staff device 200 to be communicable with the external device. The communication unit 210 is connected to the delirium occurrence predicting device 100 using wired/wireless communication to transmit and receive various data. Specifically, the communication unit 210 may receive delirium occurrence prediction data from the delirium occurrence predicting device 100.

The display unit 220 may display various interface screens to represent delirium occurrence prediction data of the individual.

According to various exemplary embodiments, the display unit 220 may include a touch screen and for example, may receive touch, gesture, proximity, drag, swipe, or hovering input which uses an electronic pen or a part of a body of the user.

The storage unit 230 may store various data used to provide a user interface to represent result data. According to various exemplary embodiments, the storage unit 230 may include at least one type of storage medium of flash memory type, hard disk type, a multimedia card micro type, and card type memories (for example, SD or XD memory and the like), a random access memory (RAM), a static random access memory (SRAM), a read only memory (ROM), an electrically erasable programmable read only memory (EEPROM), a programmable read only memory (PROM), a magnetic memory, a magnetic disk, and an optical disk.

The processor 240 is operatively connected to the communication unit 210, the display unit 220, and the storage unit 230 and may perform various instructions to provide a user interface to represent result data.

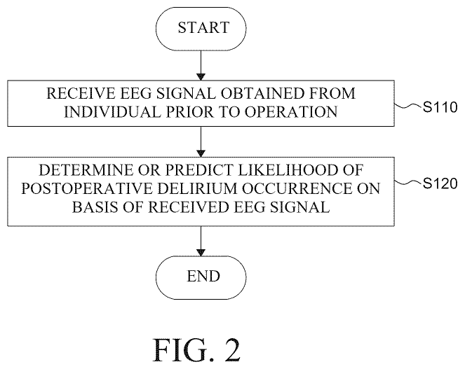

Hereinafter, a delirium occurrence predicting method according to various exemplary embodiments of the present disclosure will be described with reference to FIG. 2.

FIG. 2 is a schematic flowchart for explaining a method for providing delirium occurrence prediction data based on an EEG signal of an individual in a delirium occurrence predicting device according to an exemplary embodiment of the present disclosure.

Referring to FIG. 2, the method includes a step S110 of receiving an electroencephalography (EEG) signal obtained from an individual prior to a surgery and a step S120 of predicting a likelihood of postoperative delirium occurrence based on the received EEG signal.

To be more specific, in the step S110 of receiving the EEG signal of an individual, the EEG signal acquired in a resting state may be acquired. At this time, the individual may be an individual who does not have a history of drug use.

The delirium occurrence predicting method according to various exemplary embodiments of the present disclosure as described above allows the medical staff 250 to easily acquire delirium occurrence prediction data without having temporal and spatial constraints. Further, consistent monitoring such as treatment prognosis assessment is possible.

The EEG signal may be acquired by a fast Fourier transform (FFT) of an EEG signal having a rectangular window and the MDF value may be derived from a frequency domain analysis of an EEG signal measured for five minutes.

Further, the MDF value may be a median frequency of power values in the frequency band of 4 Hz to 15 Hz in the EEG signal. The frequency band below 4 Hz is a frequency band which is easily distorted by a safety (ElectroOculogram, EOG) noise, such as eye rolling or eye blinking and a frequency band above 15 Hz is a frequency band which is easily distorted by electromyogram (EMG) noise such as forehead muscular contraction.

To be more specific, the MDF may be a median frequency of power values in the frequency band of the EEG signal between 5.5 and 13 Hz. The ‘natural rhythm of brainwaves in a resting state with eyes closed’ appears in the frequency domain of 5.5 to 13 Hz, so that distortion caused by noises may be minimized to increase reproducibility.

The MDF value may be calculated from the acquired EEG signal by the following two steps. In a first step, all spectrum power values in the frequency domain of 5.5 to 13 Hz are added and then divided by two and in a second step, a value in the corresponding frequency at which the accumulated power of 5.5 to 13 Hz exceeds a value calculated in the first step, at the first time may be selected.

In one exemplary embodiment, when the MDF value is 8.40 or lower, it is predicted that the likelihood of postoperative delirium occurrence is high. In another exemplary embodiment, when an alpha oscillation MDF value of a frontal lobe is approximately 8.40 or lower, for example, an MDF value of (Fp1, Fp2)-frontal lobe EEG signal is approximately 8.40 or lower, the individual is classified as a risk group of delirium occurrence.

When the calculated MDF value is reduced as described above, the probability of postoperative delirium occurrence increases and for example, when an MDF value is reduced by 1, it may mean that the likelihood of postoperative delirium occurrence increases by 2.5 times.

In various exemplary embodiments, first, in the step S110 of receiving an electroencephalography (EEG) signal obtained from an individual prior to a surgery, first, an EEG signal of the individual is received and at least one feature data of an MDF, power spectrum densities (PSDs), a functional connectivity, and a network index is generated based on the EEG signal. Next, in the step S120 of predicting a likelihood of postoperative delirium occurrence based on the received EEG signal, information on a likelihood of delirium occurrence of an individual may be predicted, based on at least one feature data, by a classification model trained to output the likelihood of postoperative delirium occurrence with the feature data as an input.

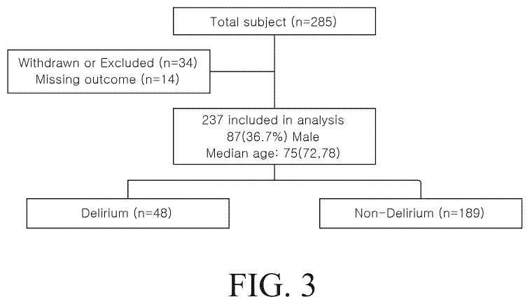

Hereinafter, a method for predicting a likelihood of postoperative delirium occurrence through a preoperative EEG signal according to an exemplary embodiment of the present disclosure will be described with reference to FIGS. 3 to 8B. FIGS. 3 to 8B illustrate an assessment result in a preoperative EEG signal of a delirium group with a postoperative delirium and a non-delirium group who does not have postoperative delirium, according to an exemplary embodiment of the present disclosure.

### Embodiment 1: Subject Selection

First, referring to FIG. 3, in this assessment, 48 postoperative delirium groups (delirium, n=48) and 189 postoperative non-delirium groups (non-delirium, n=189) were selected.

EEG signals for a total of 48 postoperative delirium groups and 189 non-delirium groups were used.

The inventors collected patient characteristics, comorbidities, and social history through patient interviews and medical chart reviews and conducted cognitive function tests on patients using mini-mental state exam (abbreviated as MMSE) and Montreal cognitive assessment (abbreviated as MoCA). The inventors collected the patient characteristics (Frailty index, global deterioration scale, instrumental activities of daily living, and mini nutritional assessment) and Charlson comorbidity index (abbreviated as CCI) through patient interviews and medical chart reviews and also collected intraoperative data (estimated blood loss, total anesthesia duration and anesthesia) along with electronic anesthesia records. Postoperative delirium was assessed at least four times a day during the postoperative hospitalization period. Delirium patients were assessed for duration of neurological symptoms, subtype of postoperative delirium, and severity of cognitive impairment using the Korean Delirium Rating Scale (K-DRS).

### Embodiment 2: EEG Method

All subjects were seated upright with their eyes closed for 5 minutes and their EEG was recorded in a resting state. In the pre-anesthesia consultation clinic prior to a surgery, EEG electrodes were applied to the subjects' frontal lobe.

During the EEG, the subjects sat comfortably in a chair under normal lighting conditions. A noninvasive monopolar scalp electrode was placed in the frontal lobe region (Fp1, Fp2 of the International 10/20 electrode system) with respect to the subject's right earlobe. A frequency pass band of a neuroNicle amplifier (LAXTHA Inc., Korea) was 3 to 43 Hz and an input range was +/−393 uV (input noise <0.6 μVrms). All filters were digital filters and IIR Butterworth filter was applied. Band stop: Second order of f1=55 Hz and f2=65 Hz. High-pass filter: First order of fc=2.6 Hz. Low-pass filter: Eighth order of fc=43 Hz. Contact impedances were maintained below 10 kΩ. All data were digitized in a continuous record mode (five minutes of EEG, sampling rate of 250 Hz, and 15 bits of resolution).

In order to minimize distorted signals such as eyeball or muscle movements, the inventors monitored the patient and EEG traces, instructed the patient to close the eyes and keep the muscles relaxed in a quiet environment, and alerted the patient whenever signs appeared.

The inventors did not arbitrarily exclude any artificial noises during the signal processing so that data contamination of (Fp1, Fp2)-frontal lobe EEG signal due to muscle and eyeball movements was tested. First, it was confirmed that none of the subjects' EEG data was contaminated by a large amount of artificial noises. In particular, none of the subjects had 10% or more of their epochs with a peak amplitude exceeding 200 uV. This value was the threshold for the exclusion criterion.

### Embodiment 3: Process of Deriving EEG Signal and MDF Value from EEG Signal

Frequency domain (or spectral domain) features are commonly used in quantitative analysis of EEG rhythms. In order to convert the EEG signal from a time domain to a frequency domain, the Fourier transform of the autocorrelation function was used, which provided the power spectral density.

EEG signals obtained from the brain at the resting stage with eyes closed are dominated by natural alpha oscillations which reflect the idle cortical state, and the dominant peak frequency is typically located in the band of 5 to 12 Hz.

The present exemplary embodiment focused on the Median Dominant Frequency (MDF) of alpha oscillation, which was a representative EEG marker explaining the phenomenon that the brain rhythm slowed down when the eyes were closed. The MDF is derived from the analysis of the 5 to 13 Hz-frequency domains of the EEG signal measured for five minutes and represents the median frequency in the dominant natural oscillation frequency band of 5.5 to 13 Hz of the EEG power spectrum. However, the exemplary embodiment is not limited thereto and the EEG power spectrum may be 4 to 15 Hz.

### Embodiment 4: Statistical Analysis and Establishment of Analysis Model

Continuous variables were presented as mean±standard deviation or median (interquartile range) and were compared using the independent t-test or Mann-Whitney U test depending on the distribution of the variables. Categorical variables were expressed as numbers (%) and were compared using the chi-square test or Fisher's exact test. Univariate logistic regression analysis (univariate analysis) was performed to identify factors associated with the risk of delirium. All multivariate logistic regression analysis (MLR) models were adjusted for age, Charlson Comorbidity Index (CCI), and anesthesia time, by considering clinical significance and statistical significance. Changes in the numerical pain rating scale over the 7 days after a surgery were compared between the two groups by a linear mixed-effect model with a compound symmetric covariance structure. The statistical analysis was performed using SAS version 9.4 and R, version 4.0.3 (http://www.r-project.org/).

### Embodiment 5: Comparison of Clinical Result of EEG Signal Between Delirium Group and Non-Delirium Group

Referring to FIG. 3, the total number of subjects enrolled during the study period was 285, 48 dropped out, and 237 were selected for the final analysis. Among them, postoperative delirium occurred in 48 subjects (20.3%). If data on the primary result were missing among enrolled patients, that information was excluded from additional analysis.

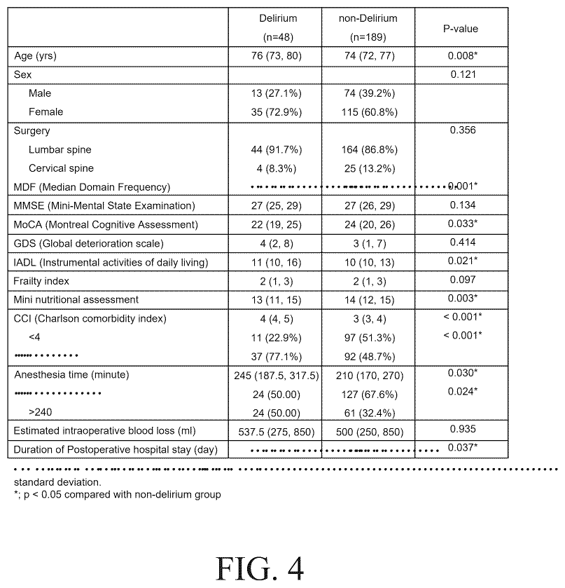

Referring to FIG. 4, the number of females (63%) exceeded the number of males (37%), and there was no difference between groups. The overall incidence of postoperative delirium was 20.3% (48 of total 237 patients). The age of the delirium group was significantly higher than that of the non-delirium group (76 vs 74, p=0.008), and there was no difference in the preoperative Mini-Mental State Examination (MMSE) between the two groups (27 vs 27, p=0.134). In contrast, the preoperative Montreal Cognitive Assessment (MoCA) score was statistically significantly lower in the delirium group than in the non-delirium group (22 vs 24, p=0.033). The MDF measured prior to the surgery was 8.65±0.69 in the delirium group and 9.02±0.61 in the non-delirium group. The MDF value of the delirium group was statistically significantly lower than the MDF value of the non-delirium group (p=0.001).

Further, compared with the non-delirium group, the delirium group had a lower mini nutritional assessment score (13 vs. 14, p=0.003), a higher Charlson comorbidity index (3 vs. 4, p<0.001), and a shorter anesthesia time (245 minutes vs. 210 minutes, p=0.030).

The mean duration of postoperative delirium was 1.98±1.81 days, and the mean delirium score assessed by the Delirium Rating Scale was 20 points. Although the peak time of postoperative delirium was known to be the third day after the surgery, in this exemplary embodiment, the occurrence time of postoperative delirium varied from immediately after surgery to one week after surgery, and the median value of occurrence was the second day after surgery.

Considering the timing and duration of postoperative delirium symptoms, the majority of patients with symptoms were observed on the fifth day after surgery.

Pain, known as the strongest risk factor for postoperative delirium, was measured repeatedly using the Numeric Rating Scale (NRS) at resting and on moving for up to 7 days after surgery (not illustrated). The NRS scores at resting were not statistically significant in terms of changes over time between groups, but the NRS scores on moving were statistically significant (p=0.044). When comparing the immediate postoperative group with the one-week postoperative group, postoperative pain persisted in the delirium group for up to one week after surgery. The total postoperative analgesic dose that affected the NRS score was converted to equivalent intravenous morphine and compared between groups, but there was no difference between groups over time (not illustrated).

### Univariate Analysis

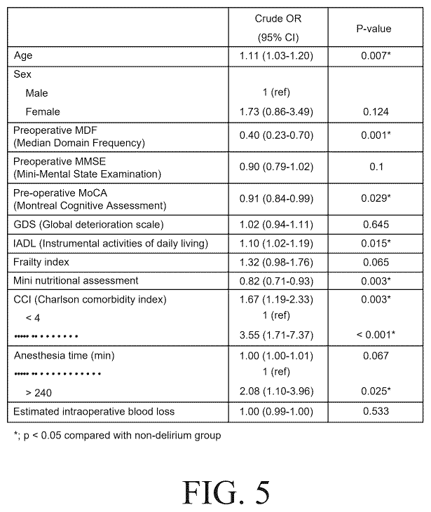

Referring to FIG. 5, in a univariate analysis of 237 subjects, numerous preoperative factors and intraoperative/postoperative patient characteristics were associated with an increased likelihood of developing postoperative delirium.

MDF was associated with postoperative delirium with an odds ratio (OR) of 0.4 [0.40 (0.23-0.70), p=0.001]. That is, it means that the lower the MDF value, the higher the probability of postoperative delirium occurrence and for every 1 decrease in the MDF value, the likelihood of postoperative delirium occurrence increases by 2.5 times. Preoperative nutritional status measured by the Mini Nutrition Index reduced the odds ratio of developing postoperative delirium [0.82 (0.71-0.93), p=0.003]. Montreal Cognitive Assessment (MoCA) scores also reduced the odds ratio of postoperative delirium [0.82 (0.71-0.93), p=0.029]. Notable patient characteristics with an odds ratio of 1 or larger were comorbidity [1.67 (1.19-2.33), p<0.001] assessed by age [1.11 (1.0.3-1.20), p=0.007] and the Charlson comorbidity index of 4 or larger. The daily living score assessed by the Instrumental activities of daily living (abbreviated as IADL) increased the probability of postoperative delirium occurrence [1.10 (1.02-1.19)].

### Multivariate Analysis

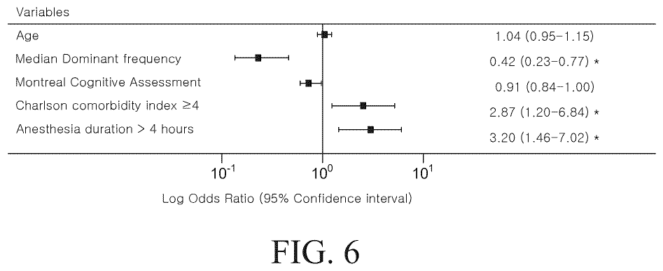

Referring to FIG. 6, in the multivariate logistic regression analysis, significant risk factors for the occurrence of postoperative delirium were the patient's age, Montreal Cognitive Frequency, a Charlson comorbidity index of 4 or higher, anesthesia duration time of 4 hours or longer, and preoperative MDF value.

Preoperative MDF was significant with an odds ratio of 0.42 (95% confidence interval 0.23 to 0.77), while Montreal Cognitive Assessment and age were not statistically significant. A Charlson comorbidity index of 4 or higher, anesthesia duration time of 4 hours or longer were statistically significant (p-value=0.018, 0.004). The goodness of fit for multivariate logistic regression was tested by the Hosmer and Lemeshaw tests (p-value=0.51).

### Result of Predicting Delirium Prior to Surgery

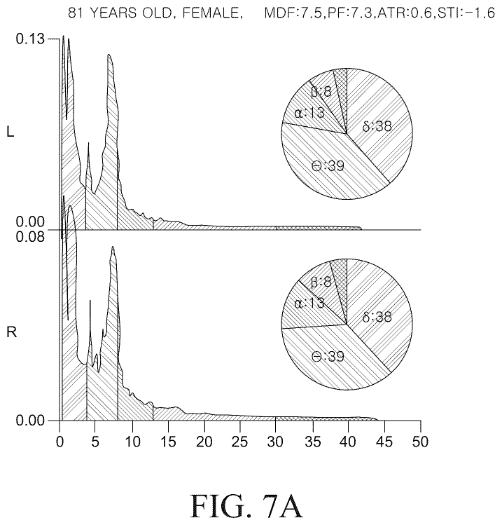

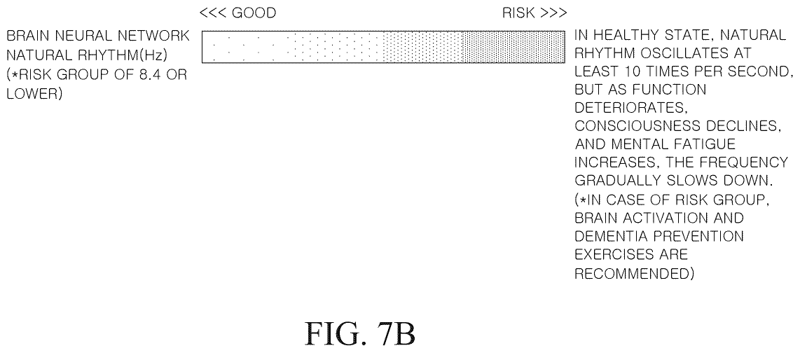

FIG. 7A illustrates a median dominant frequency (MDF) value result of power values in an EEG frequency band between 5.5 Hz and 13 Hz of one individual predicted using a delirium occurrence predicting method and device according to an exemplary embodiment of the present disclosure. Referring to FIG. 7A, the MDF value of alpha oscillation of one individual was 7.52.

Referring to FIG. 7B, delirium occurrence prediction information indicating that an individual has a high risk of postoperative delirium occurrence is provided based on the MDF value of FIG. 7A.

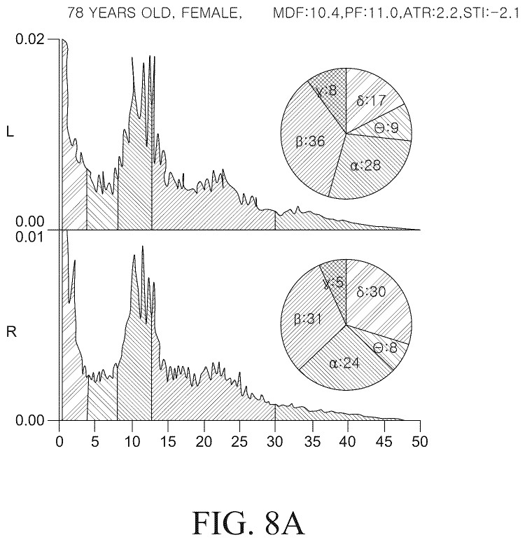

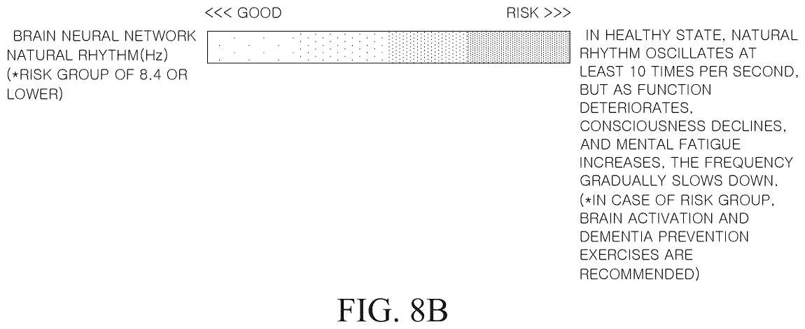

FIG. 8A illustrates a median dominant frequency (MDF) value result of power values in an EEG frequency band between 5.5 Hz and 13 Hz of another individual predicted using a delirium occurrence predicting method and device according to an exemplary embodiment of the present disclosure. Referring to FIG. 8A, an MDF value of alpha oscillation of one individual was 10.39.

Referring to FIG. 8B, delirium occurrence prediction information indicating that an individual has a low risk of postoperative delirium occurrence is provided based on the MDF value of FIG. 8A.

[National R&D Project which Supports this Disclosure]
[Project Identification Number] 1711148701
[Project Number] 2020R1A2C1012166
[Department] Ministry of Science and ICT
[Project management (Professional) Institute] National research Foundation of Korea
[Research Project] Personal basic research (Ministry of Science and ICT, R&D)
[Research Project Title] Postoperative delirium treatment strategy of elderly patient
[Contribution rate] 1/1
[Project Execution Organization] Yonsei University
[Research Period] Mar. 1, 2021, to Feb. 28, 2022

1. A delirium occurrence predicting method for predicting a likelihood of delirium occurrence prior to surgery, implemented by a processor, the method comprising the steps of:
receiving an electroencephalogram (EEG) signal obtained from an individual prior to surgery; and
predicting a likelihood of postoperative delirium occurrence based on the received EEG signal, prior to the surgery.

2. The delirium occurrence predicting method of claim 1, wherein the step of predicting the likelihood of postoperative delirium occurrence based on the received EEG signal, prior to the surgery, includes:
a step of predicting the likelihood of postoperative delirium occurrence prior to the surgery based on a power value of a frequency range of 5.5 to 13 Hz in the EEG signal.

3. The delirium occurrence predicting method of claim 1, wherein the step of predicting the likelihood of postoperative delirium occurrence based on the received EEG signal, prior to the surgery, includes:
a step of predicting the likelihood of postoperative delirium occurrence prior to the surgery based on a median dominant frequency (MDF) of power values of a frequency band of 5.5 to 13 Hz in the EEG signal.

4. The delirium occurrence predicting method of claim 3, wherein the step of predicting the likelihood of postoperative delirium occurrence based on the received EEG signal, prior to the surgery, includes:
a step of predicting that the likelihood of postoperative delirium occurrence is high when the MDF value is 8.40 or lower.

5. The delirium occurrence predicting method of claim 1, wherein in the step of receiving an EEG signal obtained from an individual prior to surgery, the EEG signal is obtained through only two EEG channels.

6. The delirium occurrence predicting method of claim 5, wherein the two EEG channels are located on a prefrontal cortex of the individual.

7. The delirium occurrence predicting method of claim 1, wherein the surgery requires general anesthesia.

8. A delirium occurrence predicting device, comprising:
a communication unit configured to receive an EEG signal obtained from an individual prior to surgery; and
a processor connected to the communication unit,
wherein the processor is configured to predict a likelihood of postoperative delirium occurrence based on the received EEG signal, prior to the surgery.

9. The delirium occurrence predicting device of claim 8, wherein the processor is further configured to predict the likelihood of postoperative delirium occurrence prior to the surgery based on a power value of a frequency range of 5.5 to 13 Hz in the EEG signal.

10. The delirium occurrence predicting device of claim 8, wherein the processor is further configured to predict the likelihood of postoperative delirium occurrence prior to the surgery based on a median dominant frequency (MDF) of power values in a frequency band of 5.5 to 13 Hz in the EEG signal.

11. The delirium occurrence predicting device of claim 10, wherein the processor is further configured to predict that when the MDF value is 8.40 or lower, the likelihood of postoperative delirium occurrence is high, prior to the surgery.

12. The delirium occurrence predicting device of claim 8, wherein the communication unit is configured to obtain the EEG signal obtained from the individual prior to the surgery through only two EEG channels.

13. The delirium occurrence predicting device of claim 12, wherein the two EEG channels are configured to be located on a prefrontal cortex of the individual.

14. The delirium occurrence predicting device of claim 8, wherein the surgery requires general anesthesia.
